(19) United States
(12) Patent Application Publication
(10) Pub. No.: US 2018/0157623 A1
(43) Pub. Date: Jun. 7, 2018

(54) SENSING DATA BASED ESTIMATION METHOD AND SENSING DATA BASED ESTIMATION SYSTEM

(71) Applicant: INSTITUTE FOR INFORMATION INDUSTRY, Taipei (TW)

(72) Inventors: Yi-Chong ZENG, Keelung City (TW); Chao-Lin WU, Changhua County (TW)

(21) Appl. No.: 15/373,383

(22) Filed: Dec. 8, 2016

(30) Foreign Application Priority Data
Dec. 1, 2016 (TW) 105139742

(51) Int. Cl. G06F 17/18; G01D 9/02; A61B 5/11; A61B 5/01; A61B 5/00

(52) U.S. Cl. CPC G06F 17/18; G01D 9/02; A61B 5/7275; A61B 5/01; A61B 5/1118

(57) ABSTRACT

A sensing data based estimation method is applied to an estimation system, and the estimation system includes a sensor, a storage and a processor. The estimation method includes operations as follows: generating a sensing data via the sensor, and the sensing data has several corresponding time parameters; receiving the sensing data and storing the sensing data to the storage via the processor, and storing a default statistical distribution via the storage in advance; executing transformation for the time parameters of the sensing data according to a default transformation relation, and executing statistical calculation for the transformed time parameters to generate a statistical set via the processor; and comparing the statistical set with the default statistical distribution, and selectively adjusting the default transformation relation according to a difference between the statistical set and the default statistical distribution via the processor, so as to generate an estimation parameter.

### RELATED APPLICATIONS

This application claims priority to Taiwan Application Serial Number 105139742, filed Dec. 1, 2016, which is herein incorporated by reference.

### BACKGROUND

### Field of Invention

The present disclosure relates to a data processing method and a data processing system. More particularly, the present disclosure relates to a sensing data based estimation method and sensing data based estimation system.

### Description of Related Art

With the rapid development of sensing technology, a sensing device is widely applied in human life and playing an increasingly important role. For example, the sensing device can be used in various fields, such as sports monitor, home living, health care, etc. However, current sensing devices in the market are unable to record user data with automatically executing data analysis. Accordingly, the sensing devices are unable to adaptively provide application service for a user. Although some sensing devices can respond an immediate user operation according to default operation modes, but this manner is still hard to adaptively provide accurate application service for the user.

Accordingly, a significant challenge is related to ways in which to effectively record and analyze user data to adaptively provide application service for a user associated with designing estimation methods and estimation systems.

### SUMMARY

An aspect of the present disclosure is directed to a sensing data based estimation method. The sensing data based estimation method is applied to an estimation system, and the estimation system includes a sensor, a storage and a processor. The estimation method includes operations as follows: generating a sensing data via the sensor, and the sensing data has a plurality of corresponding time parameters; receiving the sensing data and storing the sensing data to the storage via the processor, and storing a default statistical distribution via the storage in advance; executing transformation for the time parameters of the sensing data according to a default transformation relation, and executing statistical calculation for the transformed time parameters to generate a statistical set via the processor; and comparing the statistical set with the default statistical distribution, and selectively adjusting the default transformation relation according to a difference between the statistical set and the default statistical distribution via the processor, so as to generate an estimation parameter.

Another aspect of the present disclosure is directed to a sensing data based estimation system. The sensing data based estimation system includes a sensor, a storage and a processor. The sensor is configured to generate a sensing data, and the sensing data has several corresponding time parameters. The storage is configured to store the sensing data and a default statistical distribution. The processor is configured to execute transformation for the time parameters of the sensing data according to a default transformation relation, and to execute statistical calculation for the transformed time parameters to generate a statistical set. Then, the processor is configured to compare the statistical set with the default statistical distribution, and to selectively adjust the default transformation relation according to a difference between the statistical set and the default statistical distribution, so as to generate an estimation parameter.

It is to be understood that the foregoing general description and the following detailed description are by examples, and are intended to provide further explanation of the invention as claimed.

### BRIEF DESCRIPTION OF THE DRAWINGS

The present disclosure can be more fully understood by reading the following detailed description of the embodiment, with reference made to the accompanying drawings as follows:

FIG. 1 is a block schematic diagram of an estimation system according to embodiments of the present disclosure;

FIG. 2 is a schematic diagram of a statistical set according to embodiments of the present disclosure; and

FIG. 3 is a flow chart of an estimation method according to embodiments of the present disclosure.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

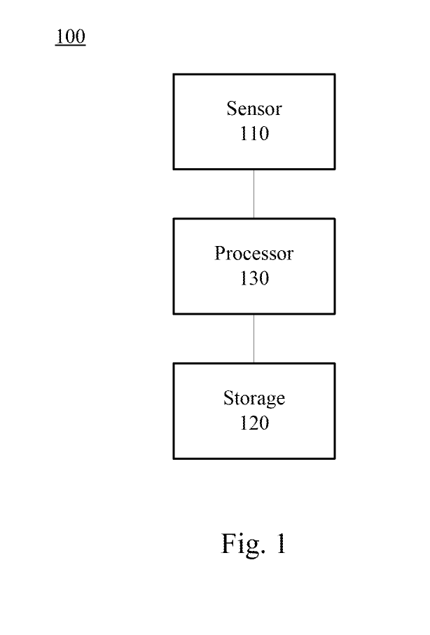

FIG. 1 is a block schematic diagram of an estimation system 100 according to embodiments of the present disclosure. As shown in FIG. 1, the estimation system 100 includes a sensor 110, a storage 120 and a processor 130. For example, the sensor 110 can be implemented by a dynamic sensor (such as, sensing a user activity or a user movement), a temperature sensor (such as, sensing a user body temperature or environmental temperature), a distance sensor (such as, sensing a user moving distance), a light sensor (such as, sensing environmental light), a counting sensor (such as, sensing the number of user activities) or any other element which can be configured to generate a sensing data, and the sensing data includes a data set of original signals detected by the sensor 110, a data set of original signals generated from the sensor 110 and a data set of processed signals; the storage 120 can be implemented by a computer hard disk, a server or any other device which can be configured to execute data storage; the processor 130 can be implemented by a central processing unit (CPU), a microcontroller or any other element which can be configured to execute data process.

The sensor 110 is configured to generate a sensing data, and the sensing data has several corresponding time parameters. For example, when a user executes particular activities, the sensor 110 can sense activity data of the user to generate the sensing data. Accordingly, the sensing data can be represented by a data set with one-dimensional elements {Δt₁, Δt₂, . . . , Δt_{N}}, or a data set with two-dimensional elements {<A₁, Δt₁>, <A₂, Δt₂>, . . . , <A_{N}, Δt_{N}>}. “Aᵢ” can represent the i-th activity executed by the user, “Δtᵢ” can represent duration of the i-th activity executed by the user, and “N” can represent a set length of the data set (that is, the number of user activity types). It should be noted that, the embodiments mentioned above are merely used for illustrating some possible manners of representing the sensing data, and the present disclosure is not limited thereto. For example, a dimension corresponding to an element of the data set can be adjusted according to practical requirements correspondingly.

The storage 120 is configured to store the sensing data and a default statistical distribution. The processor 130 is configured to execute transformation for the time parameters of the sensing data, and to execute statistical calculation for the transformed time parameters to generate a statistical set. Subsequently, the processor 130 is configured to compare the statistical set with the default statistical distribution, and to selectively adjust the default transformation relation according to a difference between the statistical set and the default statistical distribution, so as to generate an estimation parameter. For example, the default transformation relation can represent a default transformation relation corresponding to the time parameters (such as, a logarithmic function or a natural logarithmic function) or a default transformation table. Accordingly, the processor 130 can execute function transformation for the time parameters according to the default transformation function, or can execute table transformation for the time parameters according to the default transformation table. It should be noted that, the embodiments mentioned above are merely used for illustrating some possible manners of implementing the default transformation relation, and the present disclosure is not limited thereto.

In this embodiment, the default statistical distribution is stored in the storage 120 in advance. However, in some embodiments, the processor 130 can select a suitable default statistical distribution according to the sensing data, and store the selected default statistical distribution to the storage 120. Additionally, possible manners of representing the default statistical distribution can be set and adjusted according to practical requirements correspondingly. For example, the default statistical distribution can be represented by a uniform distribution, a Bernoulli distribution, a Poisson distribution, a normal distribution or any other continuous or discrete distribution. Accordingly, when the time parameters represent a first data set {10, 100, 100, 1000, 1000, 1000, 1000, 10000, 10000, 100000}, and the default statistical distribution is represented by a normal distribution, the processor 130 can transform the time parameters to a second data set {1, 2, 2, 3, 3, 3, 3, 4, 4, 5} according to a default logarithmic function (in this embodiment, that is log₁₀), and the second data set is approximate to a normal distribution. It should be noted that, the embodiments mentioned above are merely used for illustrating some possible manners of representing the default statistical distribution, and the present disclosure is not limited thereto.

In one embodiment, the processor 130 is configured to calculate a correlation between the statistical set and the default statistical distribution, and to selectively adjust the default transformation relation according to the correlation, so as to generate the estimation parameter. For example, the processor 130 can calculate the correlation between the statistical set and the default statistical distribution according to a relationship as follows:

$$\frac{{\sum }_{i = 1}^{L} D ( i ) Φ ( i )}{\sqrt{{\sum }_{i = 1}^{L} {D}^{2} ( i ) {\sum }_{i = 1}^{L} {Φ}^{2} ( i )}}$$

“D(i)” can represent the i-th statistical value of the statistical set (shown as the second data set mentioned above), and “Φ(i)” can represent the i-th statistical value of the default statistical distribution. It should be noted that, the embodiments mentioned above are merely used for illustrating some possible manners of calculating the correlation, and the present disclosure is not limited thereto. For example, the correlation can be represented by the mean square error (MSE), the minimum mean square error (MMSE) or any other parameter which can be configured to represent a difference level according to practical requirements.

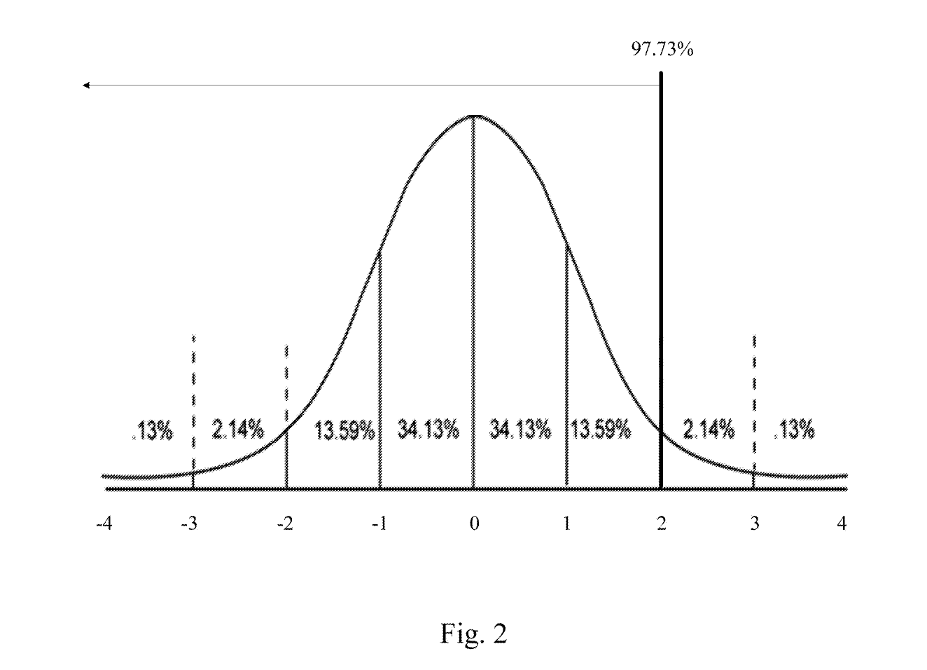

In another embodiment, when the correlation is higher than a first threshold, the processor 130 is configured to execute inverse transformation of the default transformation relation for the statistical set according to a second threshold, so as to generate the estimation parameter. For example, reference is now made to FIG. 2, and FIG. 2 is a schematic diagram of a statistical set according to embodiments of the present disclosure. As shown in FIG. 2, the second threshold can represent a confidence interval (CI) threshold, and the second threshold can be set by a user individually or by a system designer in advance. Accordingly, When a user or a system designer sets the second threshold to 97.73%, the second threshold corresponds to the transformed time parameter 2, and the processor 130 can execute the inverse transformation of the default transformation relation (in this embodiment, that is log₁₀) for the transformed time parameter 2, so as to generate the estimation parameter (that is, the time parameter 100). In other words, duration of the 97.73% activities executed by the user is lower than or equal to 100. Additionally, in some embodiments, when the duration of the activities executed by the user is temporarily or continuously higher than 100, the estimation system 100 can determine that an abnormal state is occurred (such as, a user activity period has been changed), so as to re-execute the operations mentioned above to regenerate the estimation parameter. It should be noted that, the embodiments mentioned above are merely used for illustrating some possible manners of representing the second threshold and some possible manners of calculating the estimation parameter, and the present disclosure is not limited thereto.

In further embodiment, when the correlation is lower than or equal to the first threshold, the processor 130 is configured to adjust the default transformation relation to execute re-transformation for the time parameters and regenerate the statistical set. In this embodiment, the processor 130 can adjust the default transformation relation by a manner of scaling or translating, and execute the transformation for the time parameters according to the adjusted default transformation relation, so as to regenerate the statistical set. For example, the default transformation relation can represent a default transformation relation corresponding to the time parameters or a default transformation table. Accordingly, the processor 130 can multiply (or divide) the default transformation relation and (by) a first constant (such as, A·log₁₀ or log₁₀/A, and “A” represent the first constant) to scale the default transformation relation, or add (or subtract) a second constant to (from) the default transformation relation (such as, log₁₀+B or log₁₀−B, and “B” represent the second constant) to translate the default transformation relation. It should be noted that, the embodiments mentioned above are merely used for illustrating some possible manners of adjusting the default transformation relation, and the present disclosure is not limited thereto. In further embodiment, the processor 130 is configured to calculate a correlation between the regenerated statistical set and the default statistical distribution, and to regenerate the estimation parameter according to the correlation. Since possible manners of calculating the correlation and the estimation parameter are illustrated by the embodiments mentioned above in detail, so these will not be repeated.

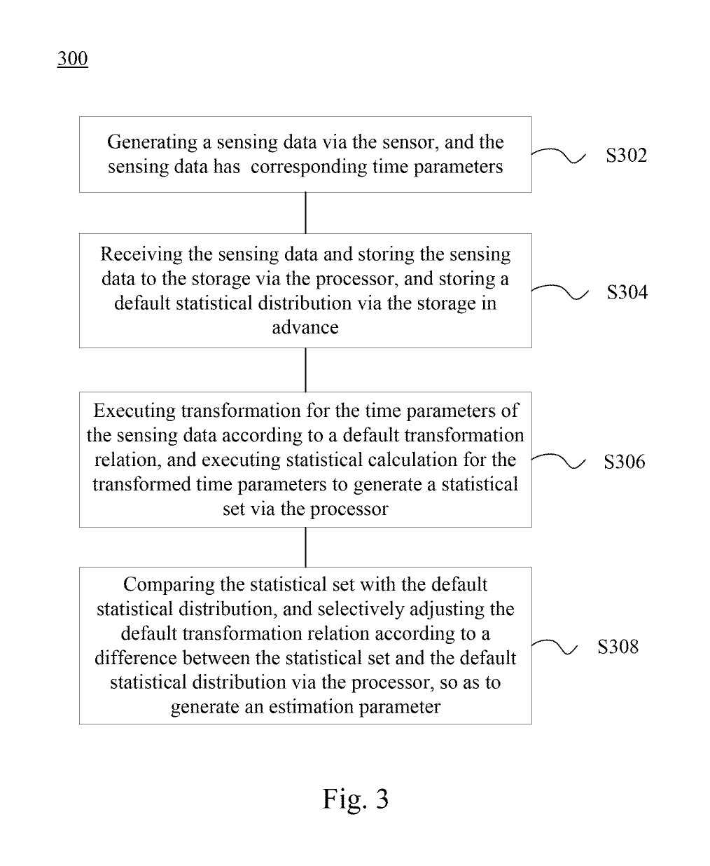

FIG. 3 is a flow chart of an estimation method 300 according to embodiments of the present disclosure. In one embodiment, the estimation method 300 can be implemented by the estimation system 100 mentioned above, but the present disclosure is not limited thereto. For facilitating of understanding the estimation method 300, the estimation system 100 is used as an example for illustrating the estimation method 300 as follows. As shown in FIG. 3, the estimation method 300 includes operations as follows:

S302: generating a sensing data via the sensor 110, and the sensing data has corresponding time parameters;

S304: receiving the sensing data and storing the sensing data to the storage 120 via the processor 130, and storing a default statistical distribution via the storage 120 in advance;

S306: executing transformation for the time parameters of the sensing data according to a default transformation relation, and executing statistical calculation for the transformed time parameters to generate a statistical set via the processor 130;

S308: comparing the statistical set with the default statistical distribution, and selectively adjusting the default transformation relation according to a difference between the statistical set and the default statistical distribution via the processor 130, so as to generate an estimation parameter.

For example, the sensor 110 can be implemented by a dynamic sensor (such as, sensing a user activity or a user movement), a temperature sensor (such as, sensing a user body temperature or environmental temperature), a distance sensor (such as, sensing a user moving distance), a light sensor (such as, sensing environmental light), a counting sensor (such as, sensing the number of user activities) or any other element which can be configured to generate a sensing data; the storage 120 described in the estimation method 300 can be implemented by a computer hard disk, a server or any other device which can be configured to execute data storage; the processor 130 described in the estimation method 300 can be implemented by a central processing unit (CPU), a microcontroller, or any other element which can be configured to execute data process.

Reference is now made to the operation S302, For example, when a user executes particular activities, the estimation method 300 can be performed by the sensor 110 to sense activity data of the user to generate the sensing data. Accordingly, the sensing data can be represent by a data set with one-dimensional elements, two-dimensional elements or multi-dimensional elements, and a dimension corresponding to an element of the data set can be adjusted according to practical requirements correspondingly. Since possible manners of representing the sensing data are illustrated by the embodiments mentioned above in detail, so these will not be repeated.

Reference is now made to the operation S304, in this embodiment, the estimation method 300 can be performed by the storage 120 to store the default statistical distribution in advance. However, in some embodiments, the estimation method 300 can be performed by the processor 130 to select a suitable default statistical distribution according to the sensing data, and to store the selected default statistical distribution to the storage 120. Additionally, possible manners of representing the default statistical distribution can be set and adjusted according to practical requirements correspondingly. For example, the default statistical distribution can be represented by a uniform distribution, a Bernoulli distribution, a Poisson distribution, a normal distribution or any other continuous or discrete distribution.

Reference is now made to the operation S306, and the default transformation relation can represent a default transformation relation corresponding to the time parameters (such as, a logarithmic function or a natural logarithmic function) or a default transformation table. Accordingly, the estimation method 300 can be performed by the processor 130 to execute function transformation for the time parameters according to the default transformation function, or to execute table transformation for the time parameters according to the default transformation table. It should be noted that, the embodiments mentioned above are merely used for illustrating some possible manners of implementing the default transformation relation, and the present disclosure is not limited thereto.

In one embodiment, reference is now made to the operation S308, and the estimation method 300 can be performed by the processor 130 to calculate a correlation between the statistical set and the default statistical distribution, and to selectively adjust the default transformation relation according to the correlation, so as to generate the estimation parameter. For example, possible manners of calculating the correlation can be referred to the relationship mentioned above, or can be represented by the mean square error (MSE), the minimum mean square error (MMSE) or any other parameter which can be configured to represent a difference level according to practical requirements.

In another embodiment, reference is now made to the operation S308, when the correlation is higher than a first threshold, the estimation method 300 can be performed by the processor 130 to execute inverse transformation of the default transformation relation for the statistical set according to a second threshold, so as to generate the estimation parameter. For example, the second threshold can represent a confidence interval (CI) threshold, and the second threshold can be set by a user individually or by a system designer in advance. Accordingly, in this embodiment, the estimation method 300 can be performed by the processor 130 to execute the inverse transformation of the default transformation relation for the transformed time parameter corresponding to the second threshold, so as to generate the estimation parameter (that is, the time parameter corresponding to the second threshold). Additionally, in some embodiments, when duration of activities executed by a user is temporarily or continuously higher than the estimation parameter, the estimation method 300 can be performed by the processor 130 to determine that an abnormal state is occurred (such as, a user activity period has been changed), so as to re-execute the operations mentioned above to regenerate the estimation parameter. It should be noted that, the embodiments mentioned above are merely used for illustrating some possible manners of representing the second threshold and some possible manners of calculating the estimation parameter, and the present disclosure is not limited thereto.

In further embodiment, reference is now made to the operation S308, when the correlation is lower than or equal to the first threshold, the estimation method 300 can be performed by the processor 130 to adjust the default transformation relation to execute re-transformation for the time parameters and regenerate the statistical set. In this embodiment, the estimation method 300 can be performed by the processor 130 to adjust the default transformation relation by a manner of scaling or translating, and to execute the transformation for the time parameters according to the adjusted default transformation relation, so as to regenerate the statistical set. For example, the default transformation relation can represent a default transformation relation corresponding to the time parameters or a default transformation table. Accordingly, the estimation method 300 can be performed by the processor 130 to multiply (or divide) the default transformation relation and (by) a first constant (such as, A·log₁₀ or log₁₀/A, and “A” represent the first constant) to scale the default transformation relation, or to add (or subtract) a second constant to (from) the default transformation relation (such as, log₁₀+B or log₁₀−B, and “B” represent the second constant) to translate the default transformation relation. It should be noted that, the embodiments mentioned above are merely used for illustrating some possible manners of adjusting the default transformation relation, and the present disclosure is not limited thereto. In further embodiments, the estimation method 300 can be performed by the processor 130 to calculate a correlation between the regenerated statistical set and the default statistical distribution, and to regenerate the estimation parameter according to the correlation. Since possible manners of calculating the correlation and the estimation parameter are illustrated by the embodiments mentioned above in detail, so these will not be repeated.

In the embodiments mentioned above, the sensing data based estimation method and the sensing data based estimation system of the present disclosure generate the sensing data via the sensor, and execute accurate data analysis according to the sensing data and the default statistical distribution via the processor, so as to generate the estimation parameter to provide suitable application service for a user. For example, the sensing data can represent user data (such as, user activity data); estimation parameter can represent analysis result of the user data (such as, a user activity period). Accordingly, the sensing data based estimation method and the sensing data based estimation system of the present disclosure can effectively record and analyze user data, so as to provide adaptive application service for a user.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the present disclosure. In view of the foregoing, it is intended that the present invention cover modifications and variations of this present disclosure provided they fall within the scope of the following claims.

What is claimed is:

1. A sensing data based estimation method, applied to an estimation system, wherein the estimation system comprises a sensor, a storage and a processor, and the estimation method comprises:
generating a sensing data via the sensor, and the sensing data comprising a plurality of corresponding time parameters;
receiving the sensing data and storing the sensing data to the storage via the processor, and storing a default statistical distribution via the storage in advance;
executing transformation for the time parameters of the sensing data according to a default transformation relation, and executing statistical calculation for the transformed time parameters to generate a statistical set via the processor; and
comparing the statistical set with the default statistical distribution, and selectively adjusting the default transformation relation according to a difference between the statistical set and the default statistical distribution via the processor, so as to generate an estimation parameter.

2. The sensing data based estimation method of claim 1, wherein comparing the statistical set with the default statistical distribution, and selectively adjusting the default transformation relation according to the difference between the statistical set and the default statistical distribution via the processor, so as to generate the estimation parameter comprises:
calculating a correlation between the statistical set and the default statistical distribution, and selectively adjusting the default transformation relation according to the correlation via the processor, so as to generate the estimation parameter.

3. The sensing data based estimation method of claim 2, wherein calculating the correlation between the statistical set and the default statistical distribution, and selectively adjusting the default transformation relation according to the correlation via the processor, so as to generate the estimation parameter comprises:
when the correlation is higher than a first threshold, executing inverse transformation of the default transformation relation for the statistical set according to a second threshold via the processor, so as to generate the estimation parameter.

4. The sensing data based estimation method of claim 2, wherein calculating the correlation between the statistical set and the default statistical distribution, and selectively adjusting the default transformation relation according to the correlation via the processor, so as to generate the estimation parameter comprises:
when the correlation is lower than or equal to the first threshold, adjusting the default transformation relation to execute re-transformation for the time parameters and regenerate the statistical set via the processor.

5. The sensing data based estimation method of claim 4, wherein calculating the correlation between the statistical set and the default statistical distribution, and selectively adjusting the default transformation relation according to the correlation via the processor, so as to generate the estimation parameter comprises:
re-calculating a correlation between the regenerated statistical set and the default statistical distribution, and regenerating the estimation parameter according to the correlation via the processor.

6. The sensing data based estimation method of claim 3, wherein calculating the correlation between the statistical set and the default statistical distribution, and selectively adjusting the default transformation relation according to the correlation via the processor, so as to generate the estimation parameter comprises:
when the correlation is lower than or equal to the first threshold, adjusting the default transformation relation to execute re-transformation for the time parameters and regenerate the statistical set via the processor.

7. The sensing data based estimation method of claim 6, wherein calculating the correlation between the statistical set and the default statistical distribution, and selectively adjusting the default transformation relation according to the correlation via the processor, so as to generate the estimation parameter comprises:
re-calculating a correlation between the regenerated statistical set and the default statistical distribution, and regenerating the estimation parameter according to the correlation via the processor.

8. The sensing data based estimation method of claim 1, wherein the sensing data comprises a data set of original signals detected by the sensor, a data set of original signals generated from the sensor and a data set of processed signals.

9. A sensing data based estimation system, comprising:
a sensor, configured to generate a sensing data, and the sensing data comprising a plurality of corresponding time parameters;
a storage, configured to store the sensing data and a default statistical distribution; and
a processor, configured to execute transformation for the time parameters of the sensing data according to a default transformation relation, and to execute statistical calculation for the transformed time parameters to generate a statistical set, wherein the processor is configured to compare the statistical set with the default statistical distribution, and to selectively adjust the default transformation relation according to a difference between the statistical set and the default statistical distribution, so as to generate an estimation parameter.

10. The sensing data based estimation system of claim 9, wherein the processor is configured to calculate a correlation between the statistical set and the default statistical distribution, and to selectively adjust the default transformation relation according to the correlation, so as to generate the estimation parameter.

11. The sensing data based estimation system of claim 10, wherein when the correlation is higher than a first threshold, the processor is configured to execute inverse transformation of the default transformation relation for the statistical set according to a second threshold, so as to generate the estimation parameter.

12. The sensing data based estimation system of claim 10, wherein when the correlation is lower than or equal to the first threshold, the processor is configured to adjust the default transformation relation to execute re-transformation for the time parameters and regenerate the statistical set.

13. The sensing data based estimation system of claim 12, wherein the processor is configured to calculate a correlation between the regenerated statistical set and the default statistical distribution, and to regenerate the estimation parameter according to the correlation.

14. The sensing data based estimation system of claim 11, wherein when the correlation is lower than or equal to the first threshold, the processor is configured to adjust the default transformation relation to execute re-transformation for the time parameters and regenerate the statistical set.

15. The sensing data based estimation system of claim 14, wherein the processor is configured to calculate a correlation between the regenerated statistical set and the default statistical distribution, and to regenerate the estimation parameter according to the correlation.

16. The sensing data based estimation system of claim 9, wherein the sensing data comprises a data set of original signals detected by the sensor, a data set of original signals generated from the sensor and a data set of processed signals.
